(19) United States
(12) Patent Application Publication
(10) Pub. No.: US 2023/0087919 A1
(43) Pub. Date: Mar. 23, 2023

(54) Cannabis Hybrid Varieties and Parent Lines

(71) Applicant: Harold Frazier, Eugene, OR (US)

(72) Inventor: Harold Frazier, Eugene, OR (US)

(21) Appl. No.: 17/933,096

(22) Filed: Sep. 17, 2022

Related U.S. Application Data
(60) Provisional application No. 63/247,682, filed on Sep. 23, 2021.

(51) Int. Cl. A01H 6/28; A01H 5/10

(52) U.S. Cl. CPC A01H 6/28; A01H 5/10

(57) ABSTRACT

The present invention discloses cannabis hybrid plant varieties designated as AT11033FL-1047, AT11043FL-1063, AT11053FL-1069, AT11063FL-1080, and AT11073FL-1081, and the inbred parent lines AT15023PS-17-2-9-15, AT15003P5-17-2-5-29, AT15013P5-18-2-2-3, AT15053P5-18-3-25-10, AT15043PS-18-3-28-19, and AT15033PS-19-1-1-11 used to produce the hybrids. The cultivars are feminized, autoflowering, cannabis varieties. Additional embodiments of the invention further provides tissues cultures, seeds, protoplasts, plant cells, and hybrids of the described cultivars.

### CROSS REFERENCE TO RELATED APPLICATIONS

This application claims priority benefit to U.S. provisional application No. 63/247,682, filed Sep. 23, 2021, the entire contents of which are hereby incorporated by reference.

### BACKGROUND OF THE INVENTION

Cannabis is a genus of flowering plants, which has been cultivated by humans for thousands of years across numerous cultures. Its fibers, flowers, seeds, and oils have been utilized for foods, medicine, and recreational purposes.

Cannabis plants produce compounds known as cannabinoids, which bind to endogenous endocannabinoid receptors. The most commonly recognized cannabinoids are delta-9 tetrahydrocannabinol (THC) and cannabidiol (CBD), although cannabis plants contain over a hundred known cannabinoids. Cannabis plants also contain a class of compounds known as terpenes, which supplement the characteristics of cannabinoids.

Research relating to the benefits of cannabis is limited due to its previous narcotic classification. There exists a need for new and improved cannabis varieties having many different permutations of cannabinoids and terpenes as the narcotic classification of cannabis diminishes. Provided herein are such improved cannabis hybrid varieties known as AT11033FL-1047, AT11043FL-1063, AT11053FL-1069, AT11063FL-1080, and AT11073FL-1081 and their inbred parents varieties AT15023PS-17-2-9-15, AT15003P5-17-2-5-29, AT15013P5-18-2-2-3, AT15053P5-18-3-25-10, AT15043P5-18-3-28-19, and AT15033PS-19-1-1-11.

### SUMMARY OF THE INVENTION

The present teachings relate to a cannabis hybrid varieties AT11033FL-1047, AT11043FL-1063, AT11053FL-1069, AT11063FL-1080, and AT11073FL-1081 and their inbred parents varieties AT15023PS-17-2-9-15, AT15003PS-17-2-5-29, AT15013PS-18-2-2-3, AT15053PS-18-3-25-10, AT15043PS-18-3-28-19, and AT15033PS-19-1-1-11 and parts and offspring thereof.

In an embodiment, a cannabis plant is provided. The plant is of a variety designated AT11033FL-1047, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT11033FL-1047, is provided. In an embodiment, a protoplast produced from the cannabis plant is provided. In an embodiment, a method of generating a processed cannabis product comprising the use of the plant is provided. In an embodiment, a cannabis product produced using the method is provided. In an embodiment, a method for producing a cannabis plant is provided wherein the method comprises crossing a cannabis plant of a variety designated AT15023PS-17-2-9-15 with a cannabis plant of a variety designated AT15003PS-17-2-5-29.

In another embodiment, a cannabis plant is provided. The plant is of a variety designated AT11053FL-1069, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT11053FL-1069, is provided. In an embodiment, a protoplast produced from the cannabis plant is provided. In an embodiment, a method of generating a processed cannabis product comprising the use of the plant is provided. In an embodiment, a cannabis product produced using the method is provided. In an embodiment, a method for producing a cannabis plant is provided wherein the method comprises crossing a cannabis plant of a variety designated AT15013PS-18-2-2-3 with a cannabis plant of a variety designated AT15053PS-18-3-25-10.

In another embodiment, a cannabis plant is provided. The plant is of a variety designated AT11073FL-1081, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT11073FL-1081, is provided. In an embodiment, a protoplast produced from the cannabis plant is provided. In an embodiment, a method of generating a processed cannabis product comprising the use of the plant is provided. In an embodiment, a cannabis product produced using the method is provided. In an embodiment, a method for producing a cannabis plant is provided wherein the method comprises crossing a cannabis plant of a variety designated AT15043PS-18-3-28-19 with a cannabis plant of a variety designated AT15033PS-19-1-1-11.

In another embodiment, a cannabis plant is provided. The plant is of a variety designated AT11043FL-1063, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT11043FL-1063, is provided. In an embodiment, a protoplast produced from the cannabis plant is provided. In an embodiment, a method of generating a processed cannabis product comprising the use of the plant is provided. In an embodiment, a cannabis product produced using the method is provided. In an embodiment, a method for producing a cannabis plant is provided wherein the method comprises crossing a cannabis plant of a variety designated AT15013PS-18-2-2-3 with a cannabis plant of a variety designated AT15043PS-18-3-28-19.

In another embodiment, a cannabis plant is provided. The plant is of a variety designated AT11063FL-1080, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT11063FL-1080, is provided. In an embodiment, a protoplast produced from the cannabis plant is provided. In an embodiment, a method of generating a processed cannabis product comprising the use of the plant is provided. In an embodiment, a cannabis product produced using the method is provided. In an embodiment, a method for producing a cannabis plant is provided wherein the method comprises crossing a cannabis plant of a variety designated AT15003PS-17-2-5-29 with a cannabis plant of a variety designated AT15033PS-19-1-1-11.

In another embodiment, a cannabis plant is provided. The plant is of a variety designated AT15023PS-17-2-9-15, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT15023PS-17-2-9-15, is provided. In an embodiment, a protoplast produced from the cannabis plant is provided. In an embodiment, a method of generating a processed cannabis product comprising the use of the plant is provided. In an embodiment, a cannabis product produced using the method is provided.

In another embodiment, a cannabis plant is provided. The plant is of a variety designated AT15003PS-17-2-5-29, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT15003PS-17-2-5-29, is provided. In an embodiment, a protoplast produced from the cannabis plant is provided. In an embodiment, a method of generating a processed cannabis product comprising the use of the plant is provided. In an embodiment, a cannabis product produced using the method is provided.

In another embodiment, a cannabis plant is provided. The plant is of a variety designated AT15013PS-18-2-2-3, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT15013PS-18-2-2-3, is provided. In an embodiment, a protoplast produced from the cannabis plant is provided. In an embodiment, a method of generating a processed cannabis product comprising the use of the plant is provided. In an embodiment, a cannabis product produced using the method is provided.

In another embodiment, a cannabis plant is provided. The plant is of a variety designated AT15053PS-18-3-25-10, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT15053PS-18-3-25-10, is provided. In an embodiment, a protoplast produced from the cannabis plant is provided. In an embodiment, a method of generating a processed cannabis product comprising the use of the plant is provided. In an embodiment, a cannabis product produced using the method is provided.

In another embodiment, a cannabis plant is provided. The plant is of a variety designated AT15043PS-18-3-28-19, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT15043PS-18-3-28-19, is provided. In an embodiment, a protoplast produced from the cannabis plant is provided. In an embodiment, a method of generating a processed cannabis product comprising the use of the plant is provided. In an embodiment, a cannabis product produced using the method is provided.

In another embodiment, a cannabis plant is provided. The plant is of a variety designated AT15033PS-19-1-1-11, wherein a representative sample of seed of said cultivar has been deposited with the American Type Culture Collection (“ATCC”). In an embodiment, the plant further comprises a trait introduced by backcrossing or genetic transformation. In an embodiment, a seed derived from the cannabis plant is provided. In an embodiment, a cannabis plant, or part thereof, including at least one plant cell, produced by growing the seed is provided. In an embodiment, an F1 hybrid seed produced by crossing the cannabis plant with a different cannabis plant is provided. In an embodiment, an F1 hybrid plant grown from the seed, or a plant part thereof, the plant part comprising at least one cell of the F1 hybrid plant, is provided. In an embodiment, an F2-F7 hybrid seed produced by crossing the F1 hybrid plant with a different cannabis plant or by a self-cross to the F1 hybrid plant is provided. In an embodiment, an F2-F7 hybrid plant grown from the seeds, or a plant part thereof, the plant part comprising at least one cell of the F2-F7 hybrid plant, is provided. In an embodiment, a tissue culture of cells produced from the cannabis plant is provided. In an embodiment, a cannabis plant generated from the tissue culture, wherein the plant has all of the morphological and physiological characteristics of variety AT15033PS-19-1-1-11, is provided. In an embodiment, a protoplast produced from the cannabis plant is provided. In an embodiment, a method of generating a processed cannabis product comprising the use of the plant is provided. In an embodiment, a cannabis product produced using the method is provided.

### BRIEF DESCRIPTION OF THE DRAWINGS

The skilled artisan will understand that the drawings, described below, are for illustration purposes only. The drawings are not intended to limit the scope of the present teachings in any way.

FIG. 1 illustrates the genetic variation, measured as proportion of homozygous sites, in samples genotyped from varieties AT11033FL-1047, AT11043FL-1063, AT11053FL-1069, AT11073FL-1081, AT15003P5-17-2-5-29, AT15013P5-18-2-2-3, AT15023P5-17-2-9-15, AT15033PS-19-1-1-11, AT15043P5-18-3-28-19, and AT15053P5-18-3-25-10.

### DETAILED DESCRIPTION OF THE INVENTION

The present teachings describe the cannabis hybrid varieties AT11033FL-1047, AT11043FL-1063, AT11053FL-1069, AT11063FL-1080, and AT11073FL-1081 and their inbred parents varieties AT15023PS-17-2-9-15, AT15003PS-17-2-5-29, AT15013PS-18-2-2-3, AT15053PS-18-3-25-10, AT15043PS-18-3-28-19, and AT15033PS-19-1-1-11. These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The terminology used in the disclosure herein is for the purpose of describing particular embodiments only and is not intended to be limiting of the disclosure. As used in the description of the embodiments of the disclosure and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, amount, dose, time, temperature, for example, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs.

### Definitions

The term “acidic cannabinoid” refers to a cannabinoid having one or more carboxylic acid functional groups. Examples of acidic cannabinoids include, but are not limited to, cannabidiolic acid (CBDA), acid tetrahydrocannabinolic acid (THCA), cannabidiolic acid (CBDA), tetrahydrocannabivarinic acid (TCHVA), and cannabichromenic acid (CBC). Acidic cannabinoids are frequently the predominant cannabinoids found in raw (i.e., unprocessed) cannabis plant material.

The term “backcrossing” refers to a process in which a breeder crosses progeny back to one of the parents one or more times, for example, a first generation hybrid F1 with one of the parental genotype of the F1 hybrid.

The term “cannabis” refers to plants of the genus Cannabis, including Cannabis sativa, Cannabis indica, and Cannabis ruderalis.

The term “cannabis oil” refers to a mixture of compounds obtained from the extraction of cannabis plants. Such compounds include, but are not limited to, cannabinoids, terpenes, terpenoids, and other compounds found in the cannabis plant. The exact composition of cannabis oil will depend on the variety of cannabis that is used for extraction, the efficiency and process of the extraction itself, and any additives that might be incorporated to alter the palatability or improve administration of the cannabis oil.

The term “cell” includes a plant cell, whether isolated, in tissue culture, or incorporated in a plant or plant part.

The term “CBD” refers to cannabidiol. The term “CBDA” refers to cannabidiolic acid.

The term “cross”, “crossing”, “cross pollination” or “cross-breeding” refer to the process by which the pollen of one flower on one plant is applied (artificially or naturally) to the ovule (stigma) of a flower on another plant. Backcrossing is a process in which a breeder repeatedly crosses hybrid progeny, for example a first generation hybrid (F1), back to one of the parents of the hybrid progeny. Backcrossing can be used to introduce one or more single locus conversions from one genetic background into another.

The term “cultivar” means a group of similar plants that by structural features and performance (e.g., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain and race are often used interchangeably by plant breeders, agronomists and farmers.

The term “donor plants” refers to the parents of a variety which contains the gene or trait of interest which is desired to be introduced into a second variety (e.g., “recipient plants”).

The term “double haploid line” refers to a stable inbred line achieved by doubling the chromosomes of a haploid line, e.g., from another culture. For example, some pollen grains (haploid) cultivated under specific conditions develop plantlets containing 1n chromosomes. The chromosomes in these plantlets are then induced to “double” (e.g., using chemical means) resulting in cells containing 2n chromosomes. The progeny of these plantlets are termed “double haploid” and are essentially not segregating any more (e.g., are stable). The term “double haploid” is used interchangeably herein with “dihaploid.”

The term “extract” refers to a solution that has been purged or dehydrated to remove residual solvent. In the methods of the invention, the extract is formed by purging or dehydrating the distillate using any known means in the art.

The term “hybrid” refers to a variety or cultivar that is the result of a cross of plants of two different varieties. A hybrid, as described here, can refer to plants that are genetically different at any particular loci. A hybrid can further include a plant that is a variety that has been bred to have at least one different characteristic from the parent, e.g., a progeny plant created from a cross between two different inbred parents wherein the hybrid progeny has at least one phenotypic characteristic that is different from each or either of the inbred parent lines. A hybrid plant can be “produced,” for example, the result of a cross between two inbred lines. A hybrid plant can also be “developed,” for example, the result of, or in the process of, research and development over the course of many generations to create a new inbred variety having distinctive characteristics. “F1 hybrid” refers to the first generation hybrid, “F2 hybrid” the second generation hybrid, “F3 hybrid” the third generation, and so on.

The term “genotype” refers to the genetic makeup of an individual cell, cell culture, tissue, organism (e.g., a plant), or group of organisms.

As used herein, the term “inbreeding” refers to the production of offspring via the mating between relatives. An “inbred line” refers to a genetically homozygous or nearly homozygous population. An inbred line, for example, can be derived through several cycles of sib crossing and/or selfing and/or via double haploid production. In some embodiments, inbred lines breed true for one or more traits of interest. An “inbred plant” or “inbred progeny” is an individual sampled from an inbred line.

As used herein, a “landrace” refers to a local variety of a domesticated plant species which has developed largely by natural processes, by adaptation to the natural and cultural environment in which it lives. The development of a landrace may also involve some selection by humans but it differs from a formal breed which has been selectively bred deliberately to conform to a particular formal, purebred standard of traits.

The term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The term “neutral cannabinoid” refers to a cannabinoid without carboxylic acid functional groups. Examples of neutral cannabinoids include, but are not limited to, THC, THCV, CBD, CBG, CBC, and CBN.

The term “offspring” refers to any plant resulting as progeny from a vegetative or sexual reproduction from one or more parent plants or descendants thereof. For instance an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait) and may include offspring with different phenotypic characteristics, while an F2 may be (and usually is) an offspring resulting from self-pollination of said F1 hybrids.

The present disclosure provides ovules and pollens of plants. As used herein when discussing plants, the term “ovule” refers to the female gametophyte, whereas the term “pollen” means the male gametophyte.

The term “plant” refers to a whole plant and any descendant, cell, tissue, or part of a plant. A class of plant that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root. A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein. In an embodiment described herein are plants in the genus of Cannabis and plants derived thereof, which can be produced asexual or sexual reproduction.

The term “plant part” or “plant tissue” or “plant material” refers to any part of a plant including but not limited to, an embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen. Plant part may also include certain extracts such as kief, oil, or hash which includes cannabis trichomes or glands.

The term “progeny” refers to any plant resulting from a vegetative or sexual reproduction from one or more parent plants or descendants thereof. For instance a progeny plant may be obtained by cloning or selfing of a parent plant or by crossing two parent plants and include selfings as well as the F1 or F2 or still further generations. An F1 is a first-generation progeny produced from parents at least one of which is used for the first time as donor of a trait, while offspring of second generation (F2) or subsequent generations (F3, F4, etc.) are specimens produced from selfings of F1's F2's etc. An F1 may thus be (and usually is) a hybrid resulting from a cross between two true breeding parents (true-breeding is homozygous for a trait) and may include F1 hybrids with new phenotypic characteristics, while an F2 may be (and usually is) an progeny resulting from self-pollination of said F1 hybrids.

The term “protoplast” as used herein refers to an entire plant cell, excluding the cell wall.

The term “sample” includes a sample from a plant, a plant part, a plant cell, or from a transmission vector, or a soil, water or air sample.

The term “secondary metabolites” as used herein refers to organic compounds that are not directly involved in the normal growth, development, or reproduction of an organism. In other words, loss of secondary metabolites does not result in immediate death of said organism.

The term “single allele converted plant” as used herein refers to those plants which are developed by a plant breeding technique called backcrossing wherein essentially all of the desired morphological and physiological characteristics of an inbred are recovered in addition to the single allele transferred into the inbred via the backcrossing technique.

The term “THC” refers to tetrahydrocannabinol. The term “THCA” refers to tetrahydrocannabinolic acid.

The term “tissue culture” refers to a composition comprising isolated cells of the same or a different type or a collection of such cells organized into parts of a plant. Exemplary types of tissue cultures are protoplasts, calli, meristematic cells, and plant cells that can generate tissue culture that are intact in plants or parts of plants, such as leaves, pollen, embryos, roots, root tips, anthers, pistils, flowers, seeds, petioles, suckers and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference

The term “transformant” refers to a cell, tissue or organism that has undergone transformation. The original transformant is designated as “T0” or “T0.” Selfing the T0 produces a first transformed generation designated as “T1” or “T1.”

The term “transformation” refers to the transfer of nucleic acid (i.e., a nucleotide polymer) into a cell. As used herein, the term “genetic transformation” refers to the transfer and incorporation of DNA, especially recombinant DNA, into a cell.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

### Cannabis

Cannabis has long been used for drug and industrial purposes, fiber (hemp), for seed and seed oils, for medicinal purposes, and for recreational purposes. Industrial hemp products are made from Cannabis plants selected to produce an abundance of fiber. Some Cannabis varieties have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Cannabis is an annual, dioecious, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

The life cycle of Cannabis varies with each variety but can be generally summarized into germination, vegetative growth, and reproductive stages. Because of heavy breeding and selection by humans, most Cannabis seeds have lost dormancy mechanisms and do not require any pre-treatments or winterization to induce germination (See Clarke, R C et al. “Cannabis: Evolution and Ethnobotany” University of California Press 2013). Seeds placed in viable growth conditions are expected to germinate in about 3 to 7 days. The first true leaves of a Cannabis plant contain a single leaflet, with subsequent leaves developing in opposite formation, with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, Cannabis plants can grow up to 2.5 inches a day, and are capable of reaching heights of up to 20 feet. Indoor growth pruning techniques tend to limit Cannabis size through careful pruning of apical or side shoots.

Some Cannabis varieties will flower without the need for external stimuli, most varieties have an absolute requirement for inductive photoperiods in the form of short days or long nights to induce fertile flowering. The first sign of flowering in Cannabis is the appearance of undifferentiated flower primordial along the main stem of the nodes. At this stage, the sex of the plants are still not distinguishable. As the flower primordia continue to develop, female (pistillate), and male (staminate) flowers can be distinguished.

For most cannabinoid producing purposes, only female plants are desired. The presence of male flowers is considered undesirable as pollination is known to reduce the cannabinoid yield, and potentially ruin a crop. For this reason, most Cannabis is grown “sinsemilla” through vegetative (i.e., asexual) propagation. In this way, only female plants are produced and no space is wasted on male plants.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al, “The draft genome and transcriptome of Cannabis sativa” Genome Biology 12:R102).

All known varieties of Cannabis are wind-pollinated and the fruit is an achene. Most varieties of Cannabis are short day plants, with the possible exception of C. sativa subsp. sativa var. spontanea (=C. ruderalis), which is commonly described as “auto-flowering” and may be day-neutral.

The genus Cannabis was formerly placed in the Nettle (Urticaceae) or Mulberry (Moraceae) family, and later, along with the Humulus genus (hops), in a separate family, the hemp family (Cannabaceae sensu stricto). Recent phylogenetic studies based on cpDNA restriction site analysis and gene sequencing strongly suggest that the Cannabaceae sensu strict arose from within the former Celtidaceae family, and that the two families should be merged to form a single monophyletic family, the Cannabaceae sensu lato.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the Cannabis plant (Rudolf Brenneisen, 2007, Chemistry and Analysis of Phytocannabinoids (cannabinoids produced produced by Cannabis) and other Cannabis Constituents, In Marijuana and the Cannabinoids, ElSohly, ed.; incorporated herein by reference) and at least 85 different cannabinoids have been isolated from the plant (EI-Alfy, Abir T, et al., 2010, “Antidepressant-like effect of delta-9-tetrahydrocannabinol and other cannabinoids isolated from Cannabis sativa L”, Pharmacology Biochemistry and Behavior 95 (4): 434-42; incorporated herein by reference). The two cannabinoids usually produced in greatest abundance are cannabidiol (CBD) and/or Δ⁹-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

Hemp is non-psychoactive and legally defined in the United States as Cannabis having less than 0.3% total THC (THCA+THC). For instruments that detect THCA levels without heat, e.g., HPLC, the THCA is not decarboxylated. Consequently, total THC levels using HPLC can be determined by multiplying the THCA levels by 87.7%, which equals the molecular weight of THCA minus its carboxyl group. For instruments that detect THCA levels using heat, e.g., gas chromatography, complete decarboxylation is presumed to have occurred and consequently, total THC is determined without multiplying THCA levels by 87.7%. Hemp's utility can be refined into a variety of commercial and industrial items, including paper, textiles, clothing, biodegradable plastics, paint, insulation, biofuel, food, and animal feed. Hemp can also be used to produce CBD.

Cannabinoids are the most studied group of secondary metabolites in Cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

Detection of neutral and acidic forms of cannabinoids are dependent on the detection method utilized. Two popular detection methods are high-performance liquid chromatography (HPLC) and gas chromatography (GC). HPLC separates, identifies, and quantifies different components in a mixture, and passes a pressurized liquid solvent containing the sample mixture through a column filled with a solid adsorbent material. Each molecular component in a sample mixture interacts differentially with the adsorbent material, thus causing different flow rates for the different components and therefore leading to separation of the components. In contrast, GC separates components of a sample through vaporization. The vaporization required for such separation occurs at high temperature. Thus, the main difference between GC and HPLC is that GC involves thermal stress and mainly resolves analytes by boiling points while HPLC does not involve heat and mainly resolves analytes by polarity. The consequence of utilizing different methods for cannabinoid detection therefore is that HPLC is more likely to detect acidic cannabinoid precursors, whereas GC is more likely to detect decarboxylated neutral cannabinoids.

The cannabinoids in cannabis plants include, but are not limited to, Δ9-Tetrahydrocannabinol (Δ⁹-THC), Δ⁸-Tetrahydrocannabinol (Δ⁸-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), Δ⁹-Tetrahydrocannabivarin (THCV), cannabichromevarin (CBCV), and cannabigerovarin (CBGV). See Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779), each of which is herein incorporated by reference in its entirety for all purposes. Non-THC cannabinoids can be collectively referred to as “CBs”, wherein CBs can be one of THCV, CBDV, CBGV, CBCV, CBD, CBC, CBE, CBG, CBN, CBND, and CBT cannabinoids.

### The Cultivars

The present invention describes numerous embodiments of the cannabis hybrid varieties AT11033FL-1047, AT11043FL-1063, AT11053FL-1069, AT11063FL-1080, and AT11073FL-1081 and their inbred parent varieties AT15023PS-17-2-9-15, AT15003P5-17-2-5-29, AT15013P5-18-2-2-3, AT15053P5-18-3-25-10, AT15043PS-18-3-28-19, and AT15033PS-19-1-1-11. In an embodiment, seeds of the cannabis hybrid varieties AT11033FL-1047, AT11043FL-1063, AT11053FL-1069, AT11063FL-1080, and AT11073FL-1081 and their inbred parent varieties AT15023PS-17-2-9-15, AT15003P5-17-2-5-29, AT15013P5-18-2-2-3, AT15053P5-18-3-25-10, AT15043PS-18-3-28-19, and AT15033PS-19-1-1-11 is provided. In an embodiment, cannabis plants are provided that comprise at least one plant cell produced by growing the seed of the cannabis hybrid varieties AT11033FL-1047, AT11043FL-1063, AT11053FL-1069, AT11063FL-1080, and AT11073FL-1081 and their inbred parent varieties AT15023P5-17-2-9-15, AT15003P5-17-2-5-29, AT15013P5-18-2-2-3, AT15053P5-18-3-25- 10, AT15043PS-18-3-28-19, and AT15033PS-19-1-1-11. In an embodiment F1 hybrid seeds are provided. The hybrid seeds are produced by crossing the cannabis hybrid varieties AT11033FL-1047, AT11043FL-1063, AT11053FL-1069, AT11063FL-1080, and AT11073FL-1081 and their inbred parent varieties AT15023PS-17-2-9-15, AT15003P5-17-2-5-29, AT15013P5-18-2-2-3, AT15053P5-18-3-25-10, AT15043PS-18-3-28-19, and AT15033PS-19-1-1-11 with different cannabis plants. In an embodiment, an F1 hybrid plant or plant part grown from a seed of an F1 hybrid of the cannabis hybrid varieties AT11033FL-1047, AT11043FL-1063, AT11053FL-1069, AT11063FL-1080, and AT11073FL-1081 and their inbred parent varieties AT15023P5-17-2-9-15, AT15003P5-17-2-5-29, AT15013P5-18-2-2-3, AT15053P5-18-3-25- 10, AT15043PS-18-3-28-19, and AT15033PS-19-1-1-11 is provided. In an embodiment a tissue culture of cells is provided. The tissue culture of cells is produced from the cannabis hybrid varieties AT11033FL-1047, AT11043FL-1063, AT11053FL-1069, AT11063FL-1080, and AT11073FL-1081 and their inbred parent varieties AT15023PS-17-2-9-15, AT15003P5-17-2-5-29, AT15013P5-18-2-2-3, AT15053P5-18-3-25-10, AT15043PS-18-3-28-19, and AT15033PS-19-1-1-11. In an embodiment a protoplast is provided. The protoplast is produced from the cannabis hybrid varieties AT11033FL-1047, AT11043FL-1063, AT11053FL-1069, AT11063FL-1080, and AT11073FL-1081 and their inbred parent varieties AT15023PS-17-2-9-15, AT15003PS-17-2-5-29, AT15013PS-18-2-2-3, AT15053PS-18-3-25-10, AT15043PS-18-3-28-19, and AT15033PS-19-1-1-11. In an embodiment a method of generating processed cannabis is provided. The method comprises the use of a plant of the the cannabis hybrid varieties AT11033FL-1047, AT11043FL-1063, AT11053FL-1069, AT11063FL-1080, and AT11073FL-1081 and their inbred parent varieties AT15023P5-17-2-9-15, AT15003P5-17-2-5-29, AT15013P5-18-2-2-3, AT15053P5-18-3-25- 10, AT15043PS-18-3-28-19, and AT15033PS-19-1-1-11. In an embodiment a cannabis product is provided. The cannabis product is produced using a plant of the the cannabis hybrid varieties AT11033FL-1047, AT11043FL-1063, AT11053FL-1069, AT11063FL-1080, and AT11073FL-1081 and their inbred parent varieties AT15023PS-17-2-9-15, AT15003P5-17-2-5-29, AT15013P5-18-2-2-3, AT15053P5-18-3-25-10, AT15043P5-18-3-28-19, and AT15033PS-19-1-1-11.

### Cannabis Inbred Parent Variety AT15023PS-17-2-9-15

Cannabis inbred parent variety AT15023PS-17-2-9-15 was developed by intercrossing 20 widely distributed and commercially available autoflowering lines. Selected plants were self-pollinated to create 29 F2 seed lots (16-2-11). Plot 16-2-11-29 was selected for high THC content. Plants were self-pollinated from 16-2-11-29 to create 28 F3 seed lots (7-2-9). Plot AT15023PS-17-2-9-15 were further inbred to create the inbred parent line AT15023PS-17-2-9-15.

Cannabis inbred parent variety AT15023P5-17-2-9-15 is a sexually propagated and feminized autoflowering variety having a short (approximately 40 cm) plant height at flowering. The branching is strong, the cotyledon shape is medium obovate, and the cotyledon color is medium green. The hypocotyl intensity of anthocyanin coloration is weak. The main stem is medium green, the main stem length of internode is short and has a mean of 3.2 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 4.1 cm. The leaf anthocyanin color in petiole is medium, and the number of leaflets is medium at 7. The central leaflet length is medium, with a mean of 9.4 cm. The central leaflet width is broad with a mean of 20.9 mm. The seed shape is ovate, the seed color of testa is grey brown, and the seed marbling of color is medium.

### Cannabis Inbred Parent Variety AT15003PS-17-2-5-29

Cannabis inbred parent variety AT15003PS-17-2-5-29 was developed by selecting seeds from a widely distributed and commercially available autoflowering line. Selected plants were self-pollinated and created 32 51 seed lots (17-2-5). Plot AT15003PS-17-2-5-29 was selected for uniformity, bud density, high THC content, compactness, and lack of genetic defects. This lot was backcrossed to reduce genetic variation and create the inbred parent line AT15003P5-17-2-5-29.

Cannabis inbred parent variety AT15003PS-17-2-5-29 is a sexually propagated and feminized autoflowering variety having a short (approximately 36 cm) plant height at flowering. The branching is strong, the cotyledon shape is medium obovate, and the cotyledon color is medium green. The hypocotyl intensity of anthocyanin coloration is weak. The main stem is medium green, the main stem length of internode is short and has a mean of 4 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 4.1 cm. The leaf anthocyanin color in petiole is medium, and the number of leaflets is medium at 7. The central leaflet length is medium, with a mean of 8.4 cm. The central leaflet width is broad with a mean of 19.9 mm. The seed shape is ovate, the seed color of testa is grey brown, and the seed marbling of color is medium.

### Cannabis Inbred Parent Variety AT15013PS-18-2-2-3

Cannabis inbred parent variety AT15013PS-18-2-2-3 was developed by intercrossing 20 widely distributed and commercially available autoflowering lines. Selected plants were self-pollinated to create 36 F2 seed lots (17-1-200). Plot 17-1-200-17 was selected for high THC content and lack of genetic defects and were self-pollinated to create 24 51 seed lots (18-2-2). Plot AT15013PS-18-2-2-3 was selected for high THC content but with lower flower density, good line uniformity, low aroma, and large calyxes. Plot AT15013PS-18-2-2-3 was further inbred to create the inbred parent line AT15013PS-18-2-2-3.

Cannabis inbred parent variety AT15013PS-18-2-2-3 is a sexually propagated and feminized autoflowering variety having a short (approximately 68 cm) plant height at flowering. The branching is strong, the cotyledon shape is medium obovate, and the cotyledon color is medium green. The hypocotyl intensity of anthocyanin coloration is weak. The main stem is medium green, the main stem length of internode is short and has a mean of 8.5 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 4.9 cm. The leaf anthocyanin color in petiole is absent or weak, and the number of leaflets is medium at 7. The central leaflet length is medium, with a mean of 10.5 cm. The central leaflet width is broad with a mean of 19.3 mm. The seed shape is ovate, the seed color of testa is grey brown, and the seed marbling of color is medium.

### Cannabis Inbred Parent Variety AT15053PS-18-3-25-10

Cannabis inbred parent variety AT15053P5-18-3-25-10 was developed by crossing seeds from widely distributed and commercially available autoflowering lines. F1 plants from the resulting cross were self-pollinated to create 11 F2 seed lots (17-1-207). Plot 17-1-207-2 was selected for high THC content and lack of genetic defects, and plants from this plot were self-pollinated to create 15 F3 seed lots. Plot 18-1-306-1 was selected for low branching, high THC content, and good flower density, and were self-pollinated to create 14 F4 seed lots. Plot AT15053P5-18-3-25-10 were selected for lack of branching, and were backcrossed to create the inbred parent line AT15053P5-18-3-25-10.

Cannabis inbred parent variety AT15053PS-18-3-25-10 is a sexually propagated and feminized autoflowering variety having a short (approximately 36 cm) plant height at flowering. The branching is low, the cotyledon shape is medium obovate, and the cotyledon color is medium green. The hypocotyl intensity of anthocyanin coloration is weak. The main stem is medium green, the main stem length of internode is short and has a mean of 2.9 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 4.5 cm. The leaf anthocyanin color in petiole is medium, and the number of leaflets is medium at 5. The central leaflet length is broad, with a mean of 20.1 cm. The central leaflet width is broad with a mean of 19.9 mm. The seed shape is ovate, the seed color of testa is grey brown, and the seed marbling of color is medium.

### Cannabis Inbred Parent Variety AT15043PS-18-3-28-19

Cannabis inbred parent variety AT15043PS-18-3-28-19 was developed by crossing seeds from widely distributed and commercially available autoflowering lines. F1 plants from the resulting cross were self-pollinated to create 11 F2 seed lots (17-1-207). Plot 17-1-207-4 was selected for high THC content, good aroma, and lack of genetic defects, and plants from this plot were self-pollinated to create 19 F3 seed lots. Plot 18-1-307-9 was selected for aroma, high THC content, and decent leaf/flower ratios, and were self-pollinated to create 20 F4 seed lots. Plot AT15043PS-18-3-28-19 were selected for good flower density, line uniformity, high THC content, and lack of genetic defect, and were backcrossed to create the inbred parent line AT15043PS-18-3-28-19.

Cannabis inbred parent variety AT15043PS-18-3-28-19 is a sexually propagated and feminized autoflowering variety having a short (approximately 51 cm) plant height at flowering. The branching is strong, the cotyledon shape is medium obovate, and the cotyledon color is medium green. The hypocotyl intensity of anthocyanin coloration is weak. The main stem is medium green, the main stem length of internode is short and has a mean of 4.4 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 4.25 cm. The leaf anthocyanin color in petiole is absent or weak, and the number of leaflets is medium at 7. The central leaflet length is medium, with a mean of 8.9 cm. The central leaflet width is broad with a mean of 16.3 mm. The seed shape is ovate, the seed color of testa is grey brown, and the seed marbling of color is medium.

### Cannabis Inbred Parent Variety AT15033PS-19-1-1-11

Cannabis inbred parent variety AT15033P5-19-1-1-11 was developed by self-pollinating AT15023P5-17-2-9-15 to create 13 51 seed lots. Plot AT15033P5-19-1-1-11 was selected for uniformity, bud density, high THC content and lack of genetic defects, including botrytis tolerance. This lot was backcrossed to reduce genetic variation and create the inbred parent line AT15033P5-19-1-1-11.

Cannabis inbred parent variety AT15033P5-19-1-1-11 is a sexually propagated and feminized autoflowering variety having a short (approximately 31.5 cm) plant height at flowering. The branching is strong, the cotyledon shape is medium obovate, and the cotyledon color is medium green. The hypocotyl intensity of anthocyanin coloration is weak. The main stem is medium green, the main stem length of internode is short and has a mean of 3.15 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 4.5 cm. The leaf anthocyanin color in petiole is absent or weak, and the number of leaflets is medium at 7. The central leaflet length is medium, with a mean of 8.9 cm. The central leaflet width is broad with a mean of 22.7 mm. The seed shape is ovate, the seed color of testa is grey brown, and the seed marbling of color is medium.

### Cannabis Hybrid Variety AT11033FL-1047

Cannabis hybrid variety AT11033FL-1047 is a hybrid produced by crossing cannabis inbred variety AT15023P5-17-2-9-15 as described herein with cannabis inbred variety AT15003PS-17-2-5-29 as described herein.

Cannabis hybrid variety AT11033FL-1047 is a sexually propagated and feminized autoflowering variety having a short (approximately 64 cm) plant height at flowering. The branching is strong, the cotyledon shape is medium obovate, and the cotyledon color is medium green. The hypocotyl intensity of anthocyanin coloration is weak. The main stem is medium green, the main stem length of internode is short and has a mean of 5 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 6.4 cm. The leaf anthocyanin color in petiole is medium, and the number of leaflets is medium at 7. The central leaflet length is medium, with a mean of 9.7 cm. The central leaflet width is broad with a mean of 18.9 mm. The seed color of testa is grey brown, and the seed marbling of color is medium.

### Cannabis Hybrid Variety AT11053FL-1069

Cannabis hybrid variety AT11053FL-1069 is a hybrid produced by crossing cannabis inbred variety AT15013PS-18-2-2-3 as described herein with cannabis inbred variety AT15053PS-18-3-25-10 as described herein.

Cannabis hybrid variety AT11053FL-1069 is a sexually propagated and feminized autoflowering variety having a short (approximately 71 cm) plant height at flowering. The branching is strong, the cotyledon shape is medium obovate, and the cotyledon color is medium green. The hypocotyl intensity of anthocyanin coloration is weak. The main stem is medium green, the main stem length of internode is short and has a mean of 8.25 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 5.4 cm. The leaf anthocyanin color in petiole is medium, and the number of leaflets is medium at 5. The central leaflet length is medium, with a mean of 10.9 cm. The central leaflet width is broad with a mean of 25 mm. The seed shape is ovate, the seed color of testa is grey brown, and the seed marbling of color is medium.

### Cannabis Hybrid Variety AT11073FL-1081

Cannabis hybrid variety AT11073FL-1081 is a hybrid produced by crossing cannabis inbred variety AT15043PS-18-3-28-19 as described herein with cannabis inbred variety AT15033PS-19-1-1-11 as described herein.

Cannabis hybrid variety AT11073FL-1081 is a sexually propagated and feminized autoflowering variety having a short (approximately 51 cm) plant height at flowering. The branching is strong, the cotyledon shape is medium obovate, and the cotyledon color is medium green. The hypocotyl intensity of anthocyanin coloration is weak. The main stem is medium green, the main stem length of internode is short and has a mean of 4.8 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 6.3 cm. The leaf anthocyanin color in petiole is medium, and the number of leaflets is medium at 7. The central leaflet length is medium, with a mean of 10.3 cm. The central leaflet width is broad with a mean of 21.5 mm. The seed shape is ovate, the seed color of testa is grey brown, and the seed marbling of color is medium.

### Cannabis Hybrid Variety AT11043FL-1063

Cannabis hybrid variety AT11043FL-1063 is a hybrid produced by crossing cannabis inbred variety AT15013PS-18-2-2-3 as described herein with cannabis inbred variety AT15043PS-18-3-28-19 as described herein.

Cannabis hybrid variety AT11043FL-1063 is a sexually propagated and feminized autoflowering variety having a moderate (approximately 93 cm) plant height at flowering. The branching is strong, the cotyledon shape is medium obovate, and the cotyledon color is medium green. The hypocotyl intensity of anthocyanin coloration is weak. The main stem is medium green, the main stem length of internode is moderate and has a mean of 9.85 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 6.2 cm. The leaf anthocyanin color in petiole is medium, and the number of leaflets is medium at 7. The central leaflet length is medium, with a mean of 11.2 cm. The central leaflet width is broad with a mean of 25.9 mm. The seed shape is ovate, the seed color of testa is grey brown, and the seed marbling of color is medium.

### Cannabis Hybrid Variety AT11063FL-1080

Cannabis hybrid variety AT11063FL-1080 is a hybrid produced by crossing cannabis inbred variety AT15003PS-17-2-5-29 as described herein with cannabis inbred variety AT15033PS-19-1-1-11 as described herein.

Cannabis hybrid variety AT11063FL-1080 is a sexually propagated and feminized autoflowering variety having a moderate (approximately 78.5 cm) plant height at flowering. The branching is strong, the cotyledon shape is medium obovate, and the cotyledon color is medium green. The hypocotyl intensity of anthocyanin coloration is weak. The main stem is medium green, the main stem length of internode is short and has a mean of 9.3 cm. The main stem thickness is medium, the main stem depth of grooves is shallow, and the main stem pith in cross-section is medium. The plant anthocyanin coloration of crown is absent or very weak. The lead intensity of green color is medium, and the leaf length of petiole is medium. The leaf length of petiole mean is 6.6 cm. The leaf anthocyanin color in petiole is medium, and the number of leaflets is medium at 7. The central leaflet length is medium, with a mean of 9.9 cm. The central leaflet width is broad with a mean of 25.2 mm. The seed shape is ovate, the seed color of testa is grey brown, and the seed marbling of color is medium.

### Cannabis Breeding

Cannabis is an important and valuable crop. Thus, a continuing goal of Cannabis plant breeders is to develop stable, high yielding Cannabis cultivars that are agronomically sound. To accomplish this goal, the Cannabis breeder preferably selects and develops Cannabis plants with traits that result in superior cultivars. The plants described herein can be used to produce new plant varieties. In some embodiments, the plants are used to develop new, unique, and superior varieties or hybrids with desired phenotypes that are different from one or more of the parental varieties.

The development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

The invention described herein is additionally directed to methods for producing a hybrid cannabis plant and parts thereof by crossing a first inbred parent cannabis plant with a second inbred parent cannabis plant wherein the first and/or second inbred parent cannabis plant is one of AT15023PS-17-2-9-15, AT15003PS-17-2-5-29, AT15013PS-18-2-2-3, AT15053PS-18-3-25-10, AT15043PS-18-3-28-19, or AT15033PS-19-1-1-11 and the hybrid cannabis plant is one of AT11033FL-1047, AT11043FL-1063, AT11053FL-1069, AT11063FL-1080, or AT11073FL-1081. Any of the following exemplary methods using the cannabis hybrid varieties AT11033FL-1047, AT11043FL-1063, AT11053FL-1069, AT11063FL-1080, and AT11073FL-1081 and their inbred parents varieties AT15023P5-17-2-9-15, AT15003P5-17-2-5-29, AT15013P5-18-2-2-3, AT15053P5-18-3-25- 10, AT15043PS-18-3-28-19, and AT15033PS-19-1-1-11 are part of this invention: selfing, backcrosses, hybrid production, crosses to populations, double haploid production, etc. All plants produced using AT15023PS-17-2-9-15, AT15003PS-17-2-5-29, AT15013PS-18-2-2-3, AT15053PS-18-3-25-10, AT15043PS-18-3-28-19, or AT15033PS-19-1-1-11 as at least one parent is within the scope of this invention, including those developed from the cannabis hybrid varieties AT11033FL-1047, AT11043FL-1063, AT11053FL-1069, AT11063FL-1080, and AT11073FL-1081 and their inbred parents varieties AT15023PS-17-2-9-15, AT15003P5-17-2-5-29, AT15013P5-18-2-2-3, AT15053P5-18-3-25-10, AT15043PS-18-3-28-19, and AT15033PS-19-1-1-11. The cannabis hybrid varieties AT11033FL-1047, AT11043FL-1063, AT11053FL-1069, AT11063FL-1080, and AT11073FL-1081 and their inbred parents varieties AT15023PS-17-2-9-15, AT15003P5-17-2-5-29, AT15013P5-18-2-2-3, AT15053PS-18-3-25-10, AT15043PS-18-3-28-19, and AT15033PS-19-1-1-11 can be used in crosses with other, different, cannabis plants (e.g., inbred lines) to produce first generation F1 cannabis hybrid seeds and plants with desirable characteristics. The cannabis plants of the invention can also be used for transformation where exogenous transgenes are introduced and expressed by the plants of the invention or for introduction of genetic changes by gene editing or mutagenesis. Genetic variants created either through traditional breeding methods, gene editing, mutagenesis or transformation of the cultivars of the invention by any of a number of protocols known to those of skill in the art are intended to be within the scope of this invention.

Another representative method of the present invention involves producing a population of AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053PS-18-3-25-10, AT15043PS-18-3-28-19, or AT15033PS-19-1-1-11 progeny plants (e.g., diploid progeny plants), comprising crossing AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25-10, AT15043PS-18-3-28-19, or AT15033PS-19-1-1-11 with another cannabis plant, thereby producing a population of cannabis plants that, on average, derives at least 6.25%, 12.5%, 25%, 30%, 35%, 40%, 45%, 50%, 55%, 60%, 65%, 70%, 80%, 85%, 90%, 95%, 96%, 97%, 98% or 99% of its alleles (i.e., TAC) from AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25-10, AT15043PS-18-3-28-19, or AT15033PS-19-1-1-11, e.g., at least about 6.25%, 12.5%, 25%, 30%, 35%, 40%, 45%, 50%, 55%, 60%, 65%, 70%, 80%, 85%, 90%, 95%, 96%, 97%, 98% or 99% of the genetic complement of AT11033FL-1047, AT11043FL-1063, AT11053FL-1069, AT11063FL-1080, AT11073FL-1081, AT15023P5-17-2-9-15, AT15003PS-17-2-5-29, AT15013P5-18-2-2-3, AT15053P5-18-3-25-10, AT15043P5-18-3-28-19, or AT15033PS-19-1-1-11. One embodiment of this invention is a cannabis plant produced by this method and that has obtained at least 6.25%, 12.5%, 25%, 30%, 35%, 40%, 45%, 50%, 55%, 60%, 65%, 70%, 80%, 85%, 90%, 95%, 96%, 97%, 98% or 99% of its alleles (i.e., TAC) from AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25- 10, AT15043PS-18-3-28-19, or AT15033PS-19-1-1-11, and optionally may be the result of a breeding process comprising one or two breeding crosses and one or more of selfing, sibbing, backcrossing and/or double haploid techniques in any combination and any order. In embodiments, the breeding process does not include a breeding cross, and comprises selfing, sibbing, backcrossing and or double haploid technology. One of ordinary skill in the art of plant breeding would know how to evaluate the traits of two plants to determine if there is or is not a significant difference between the two traits expressed by those plants. For example, see Fehr and Walt, Principles of Cultivar Development, pp. 261-286 (1987).

First generation (F1) hybrid cannabis seeds produced by crossing a plant of the cannabis varieties AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053PS-18-3-25-10, AT15043PS-18-3-28-19, or AT15033PS-19-1-1-11 to a second cannabis plant are provided. Also provided are the F1 hybrid cannabis plants grown from hybrid seeds. A hybrid can refer to plants that are genetically different at any particular loci, and can further include a plant that is a variety that has been bred to have at least one phenotypically different characteristic from the parent, e.g., a progeny plant created from a cross between the cannabis varieties AT11033FL-1047, AT11043FL-1063, AT11053FL-1069, AT11063FL-1080, AT11073FL-1081, AT15023P5-17-2-9-15, AT15003PS-17-2-5-29, AT15013P5-18-2-2-3, AT15053P5-18-3-25-10, AT15043P5-18-3-28-19, or AT15033PS-19-1-1-11 and another plant wherein the hybrid progeny has at least one phenotypic characteristic that is different from the cannabis varieties AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25- 10, AT15043PS-18-3-28-19, or AT15033PS-19-1-1-11. F1 hybrids can be either produced or developed.

In some embodiments, a plant derived from AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25-10, AT15043PS-18-3-28-19, or AT15033PS-19-1-1-11 may include a desired added trait. In representative embodiments, a cannabis plant derived from AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25- 10, AT15043PS-18-3-28-19, or AT15033PS-19-1-1-11 comprises all of the morphological and physiological characteristics of AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25-10, AT15043P5-18-3-28-19, or AT15033PS-19-1-1-11. In some embodiments, a cannabis plant derived from AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25- 10, AT15043PS-18-3-28-19, or AT15033PS-19-1-1-11 comprises essentially all of the morphological and physiological characteristics of AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25-10, AT15043PS-18-3-28-19, or AT15033PS-19-1-1-11, with the addition of a desired added trait. Those skilled in the art will appreciate that traits can be introduced into AT11033FL-1047, AT11043FL-1063, AT11053FL-1069, AT11063FL-1080, AT11073FL-1081, AT15023P5-17-2-9-15, AT15003P5-17-2-5-29, AT15013P5-18-2-2-3, AT15053P5-18-3-25- 10, AT15043PS-18-3-28-19, or AT15033PS-19-1-1-11 by any method known in the art, e.g., plant transformation methods, conventional breeding, gene editing and/or natural or induced mutations.

Details of existing Cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011), each of which is herein incorporated by reference in its entirety for all purposes.

Pedigree selection, where both single plant selection and mass selection practices are employed, may be used for the generating varieties as described herein. Pedigree selection, also known as the “Vilmorin system of selection,” is described in Fehr, Walter; Principles of Cultivar Development, Volume I, Macmillan Publishing Co., which is hereby incorporated by reference. Pedigree breeding is used commonly for the improvement of self-pollinating crops or inbred lines of cross-pollinating crops. Two parents which possess favorable, complementary traits are crossed to produce an F1. An F2 population is produced by selfing one or several F1's or by intercrossing two F1's (sib mating). Selection of the best individuals usually begins in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

A single-seed descent procedure refers to planting a segregating population, harvesting a sample of one seed per plant, and using the one-seed sample to plant the next generation. When the population has advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The complexity of inheritance also influences the choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Molecular markers can also be used in breeding programs. Molecular markers can be designed and made, based on the genome of the plants of the present application. Non-limiting examples of molecular markers can be Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs),Single Nucleotide Polymorphisms (SNPs), Amplified Fragment Length Polymorphisms (AFLPs), and Simple Sequence Repeats (SSRs), which are also referred to as Microsatellites, etc. Methods of developing molecular markers and their applications are described by Avise (Molecular markers, natural history, and evolution, Publisher: Sinauer Associates, 2004, ISBN 0878930418, 9780878930418), Srivastava et al. (Plant biotechnology and molecular markers, Publisher: Springer, 2004, ISBN1402019114, 9781402019111), and Vienne (Molecular markers in plant genetics and biotechnology, Publisher: Science Publishers, 2003), each of which is incorporated by reference in its entirety for all purposes. Molecular markers can be used in molecular marker assisted breeding. For example, the molecular markers can be utilized to monitor the transfer of the genetic material. The transferred genetic material is a gene of interest, such as genes that contribute to one or more favorable agronomic phenotypes when expressed in a plant cell, a plant part, or a plant.

Isozyme Electrophoresis and RFLPs have been widely used to determine genetic composition. Shoemaker and Olsen, (Molecular Linkage Map of Soybean (Glycine max) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD, three classical markers and four isozyme loci. See also, Shoemaker, R. C., RFLP Map of Soybean, p 299-309, in Phillips, R. L. and Vasil, I. K., eds. DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection.

Molecular markers may also be used to identify and exclude certain sources of germplasm as parental varieties or ancestors of a plant by providing a means of tracking genetic profiles through crosses.

Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety for all purposes. Cannabis genome has been sequenced (Bakel et al., The draft genome and transcriptome of Cannabis sativa, Genome Biology, 12(10):R102, 2011). Molecular markers for Cannabis plants are described in Datwyler et al. (Genetic variation in hemp and marijuana (Cannabis sativa L.) according to amplified fragment length polymorphisms, J Forensic Sci. 2006 March; 51(2):371-5), Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10(4), 2007), Datwyler et al., (Genetic Variation in Hemp and Marijuana (Cannabis sativa L.) According to Amplified Fragment Length Polymorphisms, J Forensic Sci, March 2006, 51(2):371-375), Gilmore et al. (Isolation of microsatellite markers in Cannabis sativa L. (marijuana), Molecular Ecology Notes, 3(1):105-107, March 2003), Pacifico et al., (Genetics and marker-assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726), each of which is herein incorporated by reference in its entirety for all purposes.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

### Methods of Use

The present invention provides methods of using the Cannabis plants or any parts, any compositions, or any chemicals derived from said plants of the present invention. Cannabis oil extracts can be used in the manufacture of a pharmaceutical composition or for a medicament for treating a number of conditions.

The plants can also be used for non-medical purposes. In some embodiments the specialty Cannabis plants of the present invention can be used for recreational purposes. In some embodiments, the specialty Cannabis plants of the present invention can be used for industrial purposes. In some embodiments, the plants are used for producing food, oil, wax, resin, rope, cloth, pulp, fiber, feed for livestock, construction material, plastic and composite materials, paper, jewelry, water and soil purification materials, weed control materials, cultivation materials, textiles, clothing, biodegradable plastics, body products, health food and biofuel.

### Tissue Culture

Further reproduction of the variety can occur by tissue culture and regeneration. As is well known in the art, tissue culture of Cannabis can be used for the in vitro regeneration of a Cannabis plant. Tissue culture of various tissues of Cannabis and regeneration of plants therefrom is well known and widely published. For example, reference may be had to Teng et al., HortScience. 1992, 27: 9, 1030-1032 Teng et al., HortScience. 1993, 28: 6, 669-1671, Zhang et al., Journal of Genetics and Breeding. 1992, 46: 3, 287-290, Webb et al., Plant Cell Tissue and Organ Culture. 1994, 38: 1, 77-79, Curtis et al., Journal of Experimental Botany. 1994, 45: 279, 1441-1449, Nagata et al., Journal for the American Society for Horticultural Science. 2000, 125: 6, 669-672. It is clear from the literature that the state of the art is such that these methods of obtaining plants are, and were, “conventional” in the sense that they are routinely used and have a very high rate of success. Thus, another aspect of this invention is to provide cells which upon growth and differentiation produce Cannabis plants having the physiological and morphological characteristics of variety PBI-0227-CMV.

### Products

In an embodiment a cannabis extract or product is disclosed. The product may be any product known in the cannabis arts, and can include, but is not limited to, a kief, hashish, bubble hash, an edible product, solvent reduced oil, sludge, e-juice, or tincture. As used herein, cannabis sludges are solvent-free cannabis extracts made via multigas extraction including the refrigerant 134A, butane, iso-butane and propane in a ratio that delivers a very complete and balanced extraction of cannabinoids and essential oils.

Compositions for pulmonary administration also include, but are not limited to, dry powder compositions consisting of the powder of a cannabis oil described herein, and the powder of a suitable carrier and/or lubricant. The compositions for pulmonary administration can be inhaled from any suitable dry powder inhaler device known to a person skilled in the art. In certain instances, the compositions may be conveniently delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, maltodextrin, lecithin, agarose, xanthan gum, guar gum, sorbitol, cellulose (e.g., ethyl cellulose, microcrystalline cellulose), glycine, pectin, polyacrylates and/or calcium hydrogen phosphate, calcium sulfate, (b) lubricants; e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium or potassium salt, or effervescent mixtures; (e) wetting agents, e.g., sodium lauryl sulfate, and/or (f) absorbents, colorants, flavors and sweeteners. Tablets can be either uncoated or coated according to methods known in the art. The excipients described herein can also be used for preparation of buccal dosage forms and sublingual dosage forms (e.g., films and lozenges) as described, for example, in U.S. Pat. Nos. 5,981,552 and 8,475,832. Formulation in chewing gums as described, for example, in U.S. Pat. No. 8,722,022, is also contemplated.

Further preparations for oral administration can take the form of, for example, solutions, syrups, suspensions, and toothpastes. Liquid preparations for oral administration can be prepared by conventional means with pharmaceutically acceptable additives, for example, suspending agents, for example, sorbitol syrup, cellulose derivatives, or hydrogenated edible fats; emulsifying agents, for example, lecithin, xanthan gum, or acacia; non-aqueous vehicles, for example, almond oil, sesame oil, hemp seed oil, fish oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate.

Typical formulations for topical administration include creams, ointments, sprays, lotions, hydrocolloid dressings, and patches, as well as eye drops, ear drops, and deodorants. Cannabis oils can be administered via transdermal patches as described, for example, in U.S. Pat. Appl. Pub. No. 2015/0126595 and U.S. Pat. No. 8,449,908. Formulation for rectal or vaginal administration is also contemplated. The cannabis oils can be formulated, for example, suppositories containing conventional suppository bases such as cocoa butter and other glycerides as described in U.S. Pat. Nos. 5,508,037 and 4,933,363. Compositions can contain other solidifying agents such as shea butter, beeswax, kokum butter, mango butter, illipe butter, tamanu butter, carnauba wax, emulsifying wax, soy wax, castor wax, rice bran wax, and candelilla wax. Compositions can further include clays (e.g., Bentonite, French green clays, Fuller's earth, Rhassoul clay, white kaolin clay) and salts (e.g., sea salt, Himalayan pink salt, and magnesium salts such as Epsom salt).

The compositions set forth herein can be formulated for parenteral administration by injection, for example, by bolus injection or continuous infusion. Formulations for injection can be presented in unit dosage form, for example, in ampoules or in multi-dose containers, optionally with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure, buffers, and/or other ingredients. Alternatively, the compositions can be in powder form for reconstitution with a suitable vehicle, for example, a carrier oil, before use. In addition, the compositions may also contain other therapeutic agents or substances.

The compositions can be prepared according to conventional mixing, granulating, and/or coating methods, and contain from about 0.1 to about 75%, preferably from about 1 to about 50%, of the cannabis oil extract. In general, subjects receiving a cannabis oil composition orally are administered doses ranging from about 1 to about 2000 mg of cannabis oil. A small dose ranging from about 1 to about 20 mg can typically be administered orally when treatment is initiated, and the dose can be increased (e.g., doubled) over a period of days or weeks until the maximum dose is reached.

### Example—Genetic Analyses

Genetic analyses of cannabis hybrid varieties AT11033FL-1047, AT11043FL-1063, AT11053FL-1069, AT11063FL-1080, and AT11073FL-1081 and their inbred parents varieties AT15023P5-17-2-9-15, AT15003P5-17-2-5-29, AT15013P5-18-2-2-3, AT15053P5-18-3-25- 10, AT15043PS-18-3-28-19, and AT15033PS-19-1-1-11 was conducted at Phylos Bioscience, Inc.

The samples analyzed are shown in Table 1.

TABLE 1

| | | | Genera- |
|---|---|---|---|
| Sample ID | Reference ID | Variety | tion |
| AT11033FL-1047-5 | 201130-ED-1 | AT11033FL-1047 | F1 |
| AT11033FL-1047-6 | 201130-ED-2 | AT11033FL-1047 | F1 |
| AT11033FL-1047-7 | 201130-ED-3 | AT11033FL-1047 | F1 |
| AT11033FL-1047-8 | 201130-ED-4 | AT11033FL-1047 | F1 |
| AT15023PS-17-2-9- | 201130-ED-33 | AT15023PS-17-2-9-15 | Parent |
| 15-1 | | | |
| AT15023PS-17-2-9- | 201130-ED-34 | AT15023PS-17-2-9-15 | Parent |
| 15-2 | | | |
| AT15023PS-17-2-9- | 201130-ED-35 | AT15023PS-17-2-9-15 | Parent |
| 15-3 | | | |
| AT15003PS-17-2-5- | 201130-ED-29 | AT15003PS-17-2-5-29 | Parent |
| 29-1 | | | |
| AT15003PS-17-2-5- | 201130-ED-30 | AT15003PS-17-2-5-29 | Parent |
| 29-2 | | | |
| AT15003PS-17-2-5- | 201130-ED-31 | AT15003PS-17-2-5-29 | Parent |
| 29-3 | | | |
| AT15003PS-17-2-5- | 201130-ED-32 | AT15003PS-17-2-5-29 | Parent |
| 29-4 | | | |
| AT11053FL-1069-5 | 201130-ED-5 | AT11053FL-1069 | F1 |
| AT11053FL-1069-6 | 201130-ED-6 | AT11053FL-1069 | F1 |
| AT11053FL-1069-8 | 201130-ED-8 | AT11053FL-1069 | F1 |
| AT15013PS-18-2-2-3-1 | 201130-ED-41 | AT15013PS-18-2-2-3 | Parent |
| AT15013PS-18-2-2-3-2 | 201130-ED-42 | AT15013PS-18-2-2-3 | Parent |
| AT15013PS-18-2-2-3-3 | 201130-ED-43 | AT15013PS-18-2-2-3 | Parent |
| AT15013PS-18-2-2-3-4 | 201130-ED-44 | AT15013PS-18-2-2-3 | Parent |
| AT15053PS-18-3-25- | 201130-ED-37 | AT15053PS-18-3-25- | Parent |
| 10-1 | | 10 | |
| AT15053PS-18-3-25- | 201130-ED-38 | AT15053PS-18-3-25- | Parent |
| 10-2 | | 10 | |
| AT15053PS-18-3-25- | 201130-ED-39 | AT15053PS-18-3-25- | Parent |
| 10-3 | | 10 | |
| AT11073FL-1081-5 | 201130-ED-9 | AT11073FL-1081 | F1 |
| AT11073FL-1081-6 | 201130-ED-10 | AT11073FL-1081 | F1 |
| AT11073FL-1081-7 | 201130-ED-11 | AT11073FL-1081 | F1 |
| AT11073FL-1081-8 | 201130-ED-12 | AT11073FL-1081 | F1 |
| AT15043PS-18-3-28- | 201130-ED-49 | AT15043PS-18-3-28- | Parent |
| 19-1 | | 19 | |
| AT15043PS-18-3-28- | 201130-ED-50 | AT15043PS-18-3-28- | Parent |
| 19-2 | | 19 | |
| AT15043PS-18-3-28- | 201130-ED-51 | AT15043PS-18-3-28- | Parent |
| 19-3 | | 19 | |
| AT15043PS-18-3-28- | 201130-ED-52 | AT15043PS-18-3-28- | Parent |
| 19-4 | | 19 | |
| AT15033PS-19-1-1-11-1 | 201130-ED-45 | AT15033PS-19-1-1-11 | Parent |
| AT15033PS-19-1-1-11-2 | 201130-ED-46 | AT15033PS-19-1-1-11 | Parent |
| AT15033PS-19-1-1-11-3 | 201130-ED-47 | AT15033PS-19-1-1-11 | Parent |
| AT15033PS-19-1-1-11-4 | 201130-ED-48 | AT15033PS-19-1-1-11 | Parent |
| AT11043FL-1063-18 | 210209-SU-1 | AT11043FL-1063 | F1 |
| AT11043FL-1063-17 | 210209-SU-2 | AT11043FL-1063 | F1 |
| AT11043FL-1063-9 | 210209-SU-3 | AT11043FL-1063 | F1 |
| AT11043FL-1063-8 | 210209-SU-4 | AT11043FL-1063 | F1 |

Table 2 shows determinations were made regarding relatives present on the Phylos Galaxy (identified as individuals with pairwise PI_HAT values>=0.4).

TABLE 2

| Sample ID | Relatives on Phylos Galaxy | DST | PI_HAT |
|---|---|---|---|
| AT11053FL- | Soldier 2005: https://phylos.bio/gl344 | 0.78 | 0.5 |
| 1069-8 | Gelato 41: https://phylos.bio/g-ypsag | 0.79 | 0.5 |
| AT11073FL- | Blue Dream: https://phylos.bio/gl124 | 0.77 | 0.5 |
| 1081-8 | | | |

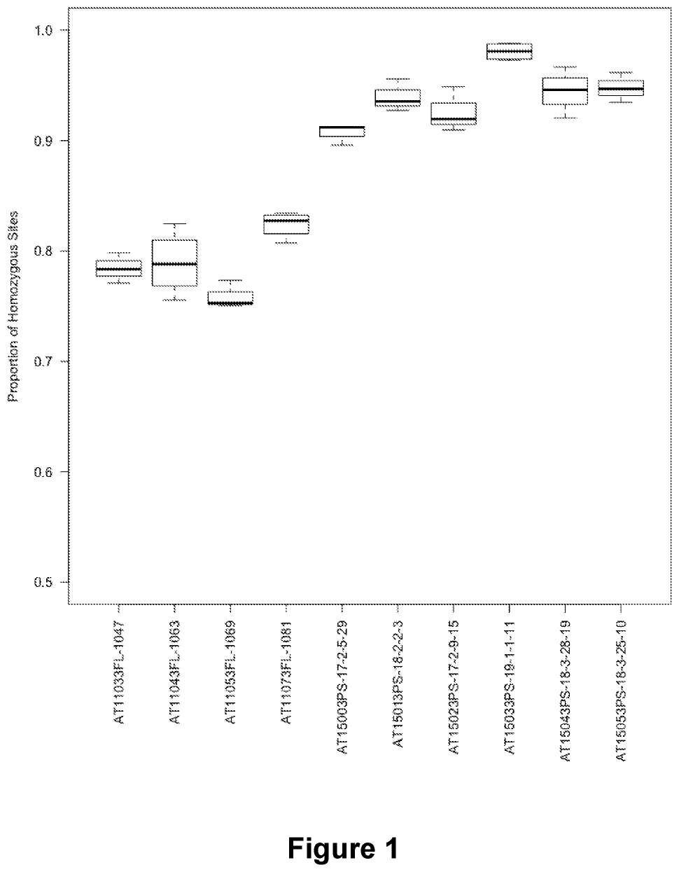

Genetic variation in samples sequenced from the AT11033FL-1047, AT11053FL-1069, and AT11073FL-1081 and their parents varieties were determined relative to samples on the Phylos Galaxy and reported as per-individual proportion of homozygous sites. FIG. 1 shows that the hybrid and parent samples exhibited higher homozygosity than Galaxy samples.

Population profiles were conducted for the AT11073FL-1081 hybrid variety and the male (AT15033PS-19-1-1-11) and female (AT15043PS-18-3-28-19) parents. The genetic background of the parent individuals was derived primarily from the Skunk and Hemp subpopulations.

Population profiles were conducted for the AT11053FL-1069 hybrid variety and the male (AT15053PS-18-3-25-10) and female (AT15013P5-18-2-2-3) parents. The genetic background of the female (AT15013PS-18-2-2-3) parents is derived primarily from the Skunk and OG Kush subpopulations, while the genetic background of the male (AT15053PS-18-3-25-10) parents are derived from Skunk and CBD subpopulations.

Population profiles were conducted for the AT11033FL-1047 hybrid variety and the male (AT15003PS-17-2-5-29) and female (AT15023PS-17-2-9-15) parents. The genetic background of the female (AT15023PS-17-2-9-15) parents is derived primarily from the Skunk, CBD, and Hemp subpopulations, while the genetic background of the male (AT15003PS-17-2-5-29) parents are derived from Skunk and Hemp subpopulations.

Distributions of pairwise DST and IBD values for each AT11053FL-1069 F1 hybrid was determined relative to all other AT11053FL-1069 F1 hybrids. Distributions of pairwise DST and IBD values for each AT11073FL-1081 F1 hybrid was determined relative to all other AT11073FL-1081 F1 hybrids. Distributions of pairwise DST and IBD values for each AT11033FL-1047 F1 hybrid was determined relative to all other AT11033FL-1047 F1 hybrids. Each F1 hybrid all showed a high degree of genetic uniformity to each respective F1 hybrid.

The pairwise genetic distance (DST) for the AT11073FL-1081 samples were conducted. F1 hybrid individuals were highly similar to one another and to parental individuals; however, the male (AT15033PS-19-1-1-11) and female (AT15043PS-18-3-28-19) parent groups were less similar to each other.

The identity by descent (IBD) values was determined for the AT11073FL-1081 samples. F1 hybrid individuals are high related to one another and to the parental individuals; however, the male (AT15033PS-19-1-1-11) and female (AT15043PS-18-3-28-19) parent groups are not related to each other.

The pairwise genetic distance (DST) for the AT11053FL-1069 samples were conducted. F1 hybrid individuals were highly similar to one another and to parental individuals; however, the male (AT15053P5-18-3-25-10) and female (AT15013PS-18-2-2-3) parent groups were less similar to each other.

The identity by descent (IBD) values was determined for the AT11053FL-1069 samples. F1 hybrid individuals are high related to one another and to the parental individuals; however, the male (AT15033PS-19-1-1-11) and female (AT15043PS-18-3-28-19) parent groups are not related to each other.

The pairwise genetic distance (DST) for the AT11033FL-1047 samples was conducted. F1 hybrid individuals were highly similar to one another and to parental individuals; however, the male (AT15003P5-17-2-5-29) and female (AT15023P5-17-2-9-15) parent groups were less similar to each other.

The identity by descent (IBD) values was determined for the AT11033FL-1047 samples. F1 hybrid individuals are high related to one another and to the parental individuals; however, the male (AT15033PS-19-1-1-11) and female (AT15043PS-18-3-28-19) parent groups are not related to each other.

### Deposit Information

A deposit of the cannabis cultivar AT11033FL-1047, which is disclosed herein above and referenced in the claims, will be made with the American Type Culture Collection (ATCC). The date of deposit is ______ and the accession number for those deposited seeds of cannabis cultivar AT11033FL-1047 is ATCC Accession No. ______.

A deposit of the cannabis cultivar AT11043FL-1063, which is disclosed herein above and referenced in the claims, will be made with the American Type Culture Collection (ATCC). The date of deposit is ______ and the accession number for those deposited seeds of cannabis cultivar AT11043FL-1063 is ATCC Accession No. ______.

A deposit of the cannabis cultivar AT11053FL-1069, which is disclosed herein above and referenced in the claims, will be made with the American Type Culture Collection (ATCC). The date of deposit is ______ and the accession number for those deposited seeds of cannabis cultivar AT11053FL-1069 is ATCC Accession No. ______.

A deposit of the cannabis cultivar AT11063FL-1080, which is disclosed herein above and referenced in the claims, will be made with the American Type Culture Collection (ATCC). The date of deposit is ______ and the accession number for those deposited seeds of cannabis cultivar AT11063FL-1080 is ATCC Accession No. ______.

A deposit of the cannabis cultivar AT11073FL-1081, which is disclosed herein above and referenced in the claims, will be made with the American Type Culture Collection (ATCC). The date of deposit is ______ and the accession number for those deposited seeds of cannabis cultivar AT11073FL-1081 is ATCC Accession No. ______.

A deposit of the cannabis cultivar AT15023PS-17-2-9-15, which is disclosed herein above and referenced in the claims, will be made with the American Type Culture Collection (ATCC). The date of deposit is ______ and the accession number for those deposited seeds of cannabis cultivar AT15023PS-17-2-9-15 is ATCC Accession No. ______.

A deposit of the cannabis cultivar AT15003PS-17-2-5-29, which is disclosed herein above and referenced in the claims, will be made with the American Type Culture Collection (ATCC). The date of deposit is ______ and the accession number for those deposited seeds of cannabis cultivar AT15003PS-17-2-5-29 is ATCC Accession No. ______.

A deposit of the cannabis cultivar AT15013PS-18-2-2-3, which is disclosed herein above and referenced in the claims, will be made with the American Type Culture Collection (ATCC). The date of deposit is ______ and the accession number for those deposited seeds of cannabis cultivar AT15013PS-18-2-2-3 is ATCC Accession No. ______.

A deposit of the cannabis cultivar AT15053PS-18-3-25-10, which is disclosed herein above and referenced in the claims, will be made with the American Type Culture Collection (ATCC). The date of deposit is ______ and the accession number for those deposited seeds of cannabis cultivar AT15053PS-18-3-25-10 is ATCC Accession No. ______.

A deposit of the cannabis cultivar AT15043PS-18-3-28-19, which is disclosed herein above and referenced in the claims, will be made with the American Type Culture Collection (ATCC). The date of deposit is ______ and the accession number for those deposited seeds of cannabis cultivar AT15043PS-18-3-28-19 is ATCC Accession No. ______.

A deposit of the cannabis cultivar AT15033PS-19-1-1-11, which is disclosed herein above and referenced in the claims, will be made with the American Type Culture Collection (ATCC). The date of deposit is ______ and the accession number for those deposited seeds of cannabis cultivar AT15033PS-19-1-1-11 is ATCC Accession No. ______.

All restrictions upon the deposits have been removed, and the deposits are intended to meet all of the requirements of the Budapest Treaty and 37 C.F.R. § 1.801-1.809. The deposits will be maintained in the depository for a period of 30 years, or 5 years after the last request, or for the effective life of the patent, whichever is longer, and will be replaced if necessary during that period. Applicant does not waive any infringement of rights granted under this patent or under the Plant Variety Protection Act (7 USC 2321 et seq.).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the invention as defined in the appended claims.

What is claimed is:

1-96. (canceled)

97. A cannabis plant of a variety designated AT15013PS-18-2-2-3, wherein a representative sample of seed of said cultivar has been deposited with the American Type Culture Collection (“ATCC”).

98. The plant of claim 97, further comprising a trait introduced by backcrossing or genetic transformation.

99. A seed derived from the cannabis plant of claim 97.

100. A cannabis plant, or part thereof, including at least one plant cell, produced by growing the seed of claim 99.

101. An F1 hybrid seed produced by crossing the cannabis plant of claim 97 with a different cannabis plant.

102. An F1 hybrid plant grown from the seed of claim 101, or a plant part thereof, the plant part comprising at least one cell of the F1 hybrid plant.

103-107. (canceled)

108. A method of generating a processed cannabis product comprising the use of a plant from claim 97.

109. A cannabis product produced using the method of claim 108.

110. A cannabis plant of a variety designated AT15053PS-18-3-25-10, wherein a representative sample of seed of said cultivar has been deposited with the American Type Culture Collection (“ATCC”).

111. The plant of claim 110, further comprising a trait introduced by backcrossing or genetic transformation.

112. A seed derived from the cannabis plant of claim 110.

113. A cannabis plant, or part thereof, including at least one plant cell, produced by growing the seed of claim 112.

114. An F1 hybrid seed produced by crossing the cannabis plant of claim 110 with a different cannabis plant.

115. An F1 hybrid plant grown from the seed of claim 114, or a plant part thereof, the plant part comprising at least one cell of the F1 hybrid plant.

116-120. (canceled)

121. A method of generating a processed cannabis product comprising the use of a plant from claim 110.

122. A cannabis product produced using the method of claim 121.

123-148. (canceled)

149. An F1 hybrid seed produced by crossing the cannabis plant of claim 97 with the cannabis plant of claim 110.

150. An F1 hybrid plant grown from the seed of claim 149, or a plant part thereof, the plant part comprising at least one cell of the F1 hybrid plant.

151. The F1 hybrid plant, or plant part thereof, of claim 150 wherein the F1 hybrid plant, or plant part thereof, is a variety designated AT11033FL-1047, wherein a representative sample of seed of said cultivar has been deposited with the American Type Culture Collection (“ATCC”).

152. The F1 hybrid plant, or plant part thereof, of claim 150, further comprising a trait introduced by backcrossing or genetic transformation.

153. A seed derived from the F1 hybrid plant of claim 150.

154. A cannabis plant, or part thereof, including at least one plant cell, produced by growing the seed of claim 153.

155. A method of generating a processed cannabis product comprising the use of F1 hybrid plant, or plant part thereof, of claim 150.

156. A cannabis product produced using the method of claim 158.

157. The product of claim 159 wherein the product is a kief, hashish, bubble hash, an edible product, colvent reduced oil, sludge, e-juice, or tincture.
